(19) United States
(12) Patent Application Publication
(10) Pub. No.: US 2019/0348180 A1
(43) Pub. Date: Nov. 14, 2019

(54) SYSTEM AND METHOD FOR PROVIDING MODEL-BASED PREDICTIONS OF PATIENT-RELATED METRICS BASED ON LOCATION-BASED DETERMINANTS OF HEALTH

(71) Applicant: KONINKLIJKE PHILIPS N.V., Eindhoven (NL)

(72) Inventors: Reza SHARIFI SEDEH, Malden, MA (US); Eran SIMHON, Boston, MA (US)

(21) Appl. No.: 16/395,455

(22) Filed: Apr. 26, 2019

Related U.S. Application Data
(60) Provisional application No. 62/669,426, filed on May 10, 2018.

(51) Int. Cl. G16H 50/20; G16H 50/50; G06N 20/00

(52) U.S. Cl. CPC G16H 50/20; G06N 20/00; G16H 50/50

(57) ABSTRACT

The present disclosure pertains to a system for providing model-based predictions of patient-related metrics based on location-based determinants of health. In some embodiments, the system (i) obtains (a) one or more patient-related features and (b) one or more location-related features associated with an individual; (ii) performs one or more queries based on the one or more patient-related features associated with the individual to obtain one or more location-related features associated with similar individuals; and (iii) provides the one or more location-related features associated with the similar individuals and the one or more location-related features associated with the individual to the machine learning model to predict (a) one or more metric values for the patient-related metrics associated with the individual and (b) at least one location-related feature associated with the individual likely to contribute to the one or more metric values.

### CROSS-REFERENCE TO PRIOR APPLICATIONS

This application claims the benefit of U.S. Patent Application No. 62/669,426, filed on 10 May 2018. This application is hereby incorporated by reference herein.

### BACKGROUND

### 1. Field

The present disclosure pertains to a system and method for providing model-based predictions of patient-related metrics based on location-based determinants of health.

### 2. Description of the Related Art

Social behavioral and environmental factors account for more premature deaths than genomics and traditional healthcare-related factors in the United States. In fact, social, behavioral, and environmental factors account for the majority of all premature deaths in the United States. Care management emphasizes prevention, continuity of care and coordination of care, which advocates for, and links individuals to, services as necessary across providers and settings. Although automated and other computer-assisted care management systems exist, such systems may often fail to properly assign patients to appropriate care plans to improve different patient-related metrics, especially given that care plan assignments rely on domain knowledge of a care manager. For example, care managers may not be properly equipped to determine the impact of the care plans on the patient-related metrics quantitatively or investigate various care plans for a particular patient. These and other drawbacks exist.

### SUMMARY

Accordingly, one or more aspects of the present disclosure relate to a system for providing model-based predictions of patient-related metrics based on location-based determinants of health. The system comprises one or more processors configured by machine readable instructions and/or other components. The one or more hardware processors are configured to: obtain (i) one or more patient-related features and (ii) one or more location-related features associated with an individual; perform, on one or more databases containing at least (i) one or more patient-related features and (ii) one or more location-related features associated with similar individuals, one or more queries based on the one or more patient-related features associated with the individual to obtain the one or more location-related features associated with the similar individuals; provide the one or more location-related features associated with the similar individuals to a machine learning model to train the machine learning model, the machine learning model configured to make predictions related to one or more patient-related metrics; and provide, subsequent to the training of the machine learning model, the one or more location-related features associated with the individual to the machine learning model to predict (a) one or more metric values for the patient-related metrics associated with the individual and (b) at least one location-related feature associated with the individual likely to contribute to the one or more metric values.

Another aspect of the present disclosure relates to a method for providing model-based predictions of patient-related metrics based on location-based determinants of health with a system. The system comprises one or more processors configured by machine readable instructions and/or other components. The method comprises: obtaining, with one or more processors, (i) one or more patient-related features and (ii) one or more location-related features associated with an individual; performing, with the one or more processors, one or more queries on one or more databases containing at least (i) one or more patient-related features and (ii) one or more location-related features associated with similar individuals, the one or more queries being based on the one or more patient-related features associated with the individual to obtain the one or more location-related features associated with the similar individuals; providing, with the one or more processors, the one or more location-related features associated with the similar individuals to a machine learning model to train the machine learning model, the machine learning model configured to make predictions related to one or more patient-related metrics; and providing, with the one or more processors, the one or more location-related features associated with the individual to the machine learning model subsequent to the training of the machine learning model to predict (a) one or more metric values for the patient-related metrics associated with the individual and (b) at least one location-related feature associated with the individual likely to contribute to the one or more metric values.

Still another aspect of present disclosure relates to a system for providing model-based predictions of patient-related metrics based on location-based determinants of health. The system comprises: means for obtaining (i) one or more patient-related features and (ii) one or more location-related features associated with an individual; means for performing one or more queries on one or more databases containing at least (i) one or more patient-related features and (ii) one or more location-related features associated with similar individuals, the one or more queries being based on the one or more patient-related features associated with the individual to obtain the one or more location-related features associated with the similar individuals; means for providing the one or more location-related features associated with the similar individuals to a machine learning model to train the machine learning model, the machine learning model configured to make predictions related to one or more patient-related metrics; and means for providing the one or more location-related features associated with the individual to the machine learning model subsequent to the training of the machine learning model to predict (a) one or more metric values for the patient-related metrics associated with the individual and (b) at least one location-related feature associated with the individual likely to contribute to the one or more metric values.

These and other objects, features, and characteristics of the present disclosure, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 is a schematic illustration of a system configured for providing model-based predictions of patient-related metrics based on location-based determinants of health, in accordance with one or more embodiments.

FIG. 2 illustrates a feature selection interface, in accordance with one or more embodiments.

FIG. 3 illustrates assignment of care plans to an individual, in accordance with one or more embodiments.

FIG. 4 illustrates a care plan suggestion interface, in accordance with one or more embodiments.

FIG. 5 illustrates a method for providing model-based predictions of patient-related metrics based on location-based determinants of health, in accordance with one or more embodiments.

### DETAILED DESCRIPTION OF EXEMPLARY EMBODIMENTS

As used herein, the singular form of “a”, “an”, and “the” include plural references unless the context clearly dictates otherwise. As used herein, the term “or” means “and/or” unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

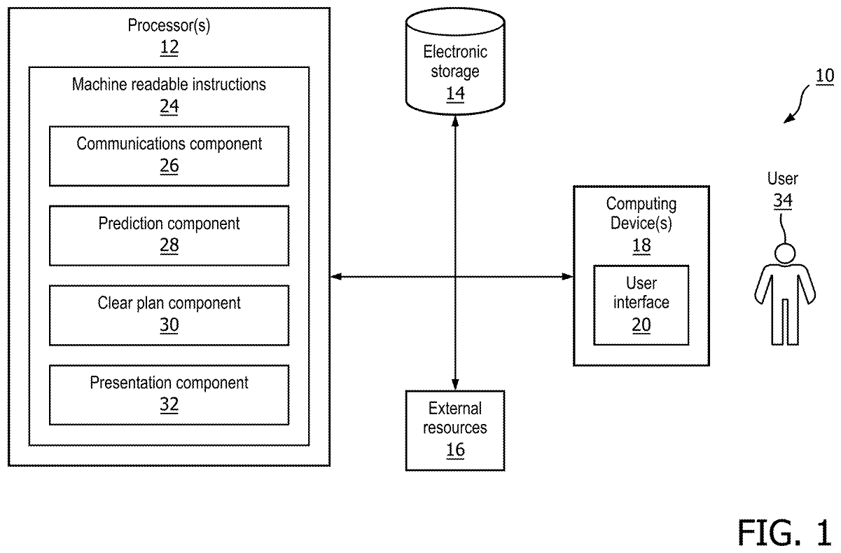

FIG. 1 is a schematic illustration of a system 10 configured for providing model-based predictions of patient-related metrics based on location-based determinants of health, in accordance with one or more embodiments. In some embodiments, system 10 is configured to facilitate, via a user interface, selection, by a user (e.g., a care manager) of patient-related features, location-related features, patient-related metrics, or other information. In some embodiments, system 10 is configured to determine social determinants of health (e.g., from the location-related features) that have a greater impact on the patient-related metrics, compared to other social determinants of health. As an example, system 10 may determine the most impactful social determinants of health on the patient-related metrics. In some embodiments, system 10 is configured to determine the patient-related metrics associated with an individual based on the social determinants of health (e.g., most impactful social determinants of health or other select social determinants of health), patient-related metrics associated with similar individuals, or other information. In some embodiments, system 10 is configured to assign different care plans to the individual, which subsequently changes the social determinants of health for the individual. In some embodiments, system 10 is configured to determine updated patient-related metrics based on the changed social determinants of health for the individual. In some embodiments, system 10 is configured to predict patient-related metrics improvements and costs associated with care plans causing the improvement.

In some embodiments, system 10 is configured to generate one or more predictions related to (a) one or more metric values for the patient-related metrics associated with an individual and (b) at least one location-related feature associated with the individual likely to contribute to the metric values, or perform other operations described herein via one or more prediction models. Such prediction models may include neural networks, other machine learning models, or other prediction models. As an example, neural networks may be based on a large collection of neural units (or artificial neurons). Neural networks may loosely mimic the manner in which a biological brain works (e.g., via large clusters of biological neurons connected by axons). Each neural unit of a neural network may be connected with many other neural units of the neural network. Such connections can be enforcing or inhibitory in their effect on the activation state of connected neural units. In some embodiments, each individual neural unit may have a summation function which combines the values of all its inputs together. In some embodiments, each connection (or the neural unit itself) may have a threshold function such that the signal must surpass the threshold before it is allowed to propagate to other neural units. These neural network systems may be self-learning and trained, rather than explicitly programmed, and can perform significantly better in certain areas of problem solving, as compared to traditional computer programs. In some embodiments, neural networks may include multiple layers (e.g., where a signal path traverses from front layers to back layers). In some embodiments, back propagation techniques may be utilized by the neural networks, where forward stimulation is used to reset weights on the “front” neural units. In some embodiments, stimulation and inhibition for neural networks may be more free-flowing, with connections interacting in a more chaotic and complex fashion.

In some embodiments, system 10 comprises processors 12, electronic storage 14, external resources 16, computing device 18 (e.g., associated with user 34), or other components.

Electronic storage 14 comprises electronic storage media that electronically stores information (e.g., location-related features associated with similar individuals). The electronic storage media of electronic storage 14 may comprise one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 14 may be (in whole or in part) a separate component within system 10, or electronic storage 14 may be provided (in whole or in part) integrally with one or more other components of system 10 (e.g., computing device 18, etc.). In some embodiments, electronic storage 14 may be located in a server together with processors 12, in a server that is part of external resources 16, and/or in other locations. Electronic storage 14 may comprise one or more of optically readable storage media (e.g., optical disks, etc.), magnetically readable storage media (e.g., magnetic tape, magnetic hard drive, floppy drive, etc.), electrical charge-based storage media (e.g., EPROM, RAM, etc.), solid-state storage media (e.g., flash drive, etc.), and/or other electronically readable storage media. Electronic storage 14 may store software algorithms, information determined by processors 12, information received via processors 12 and/or graphical user interface 20 and/or other external computing systems, information received from external resources 16, and/or other information that enables system 10 to function as described herein.

External resources 16 include sources of information and/or other resources. For example, external resources 16 may include a population's electronic medical record (EMR), the population's electronic health record (EHR), or other information. In some embodiments, external resources 16 include health information related to the population. In some embodiments, the health information comprises demographic information, vital signs information, medical condition information indicating medical conditions experienced by individuals in the population, treatment information indicating treatments received by the individuals, care management information, and/or other health information. In some embodiments, external resources 16 include sources of information such as databases, websites, etc., external entities participating with system 10 (e.g., a medical records system of a health care provider that stores medical history information of patients), one or more servers outside of system 10, and/or other sources of information. In some embodiments, external resources 16 include components that facilitate communication of information such as a network (e.g., the internet), electronic storage, equipment related to Wi-Fi technology, equipment related to Bluetooth® technology, data entry devices, sensors, scanners, and/or other resources. In some embodiments, some or all of the functionality attributed herein to external resources 16 may be provided by resources included in system 10.

Processors 12, electronic storage 14, external resources 16, computing device 18, and/or other components of system 10 may be configured to communicate with one another, via wired and/or wireless connections, via a network (e.g., a local area network and/or the internet), via cellular technology, via Wi-Fi technology, and/or via other resources. It will be appreciated that this is not intended to be limiting, and that the scope of this disclosure includes embodiments in which these components may be operatively linked via some other communication media. In some embodiments, processors 12, electronic storage 14, external resources 16, computing device 18, and/or other components of system 10 may be configured to communicate with one another according to a client/server architecture, a peer-to-peer architecture, and/or other architectures.

Computing device 18 may be configured to provide an interface between user 34 and/or other users, and system 10. In some embodiments, computing device 18 is and/or is included in desktop computers, laptop computers, tablet computers, smartphones, smart wearable devices including augmented reality devices (e.g., Google Glass), wrist-worn devices (e.g., Apple Watch), and/or other computing devices associated with user 34, and/or other users. In some embodiments, computing device 18 facilitates presentation of a suggested list of care plans, the projected patient-related metrics improvements and corresponding costs of care plans, or other information. Accordingly, computing device 18 comprises a user interface 20. Examples of interface devices suitable for inclusion in user interface 20 include a touch screen, a keypad, touch sensitive or physical buttons, switches, a keyboard, knobs, levers, a camera, a display, speakers, a microphone, an indicator light, an audible alarm, a printer, tactile haptic feedback device, or other interface devices. The present disclosure also contemplates that computing device 18 includes a removable storage interface. In this example, information may be loaded into computing device 18 from removable storage (e.g., a smart card, a flash drive, a removable disk, etc.) that enables caregivers or other users to customize the implementation of computing device 18. Other exemplary input devices and techniques adapted for use with computing device 18 or the user interface include an RS-232 port, RF link, an IR link, a modem (telephone, cable, etc.), or other devices or techniques.

Processor 12 is configured to provide information processing capabilities in system 10. As such, processor 12 may comprise one or more of a digital processor, an analog processor, a digital circuit designed to process information, an analog circuit designed to process information, a state machine, or other mechanisms for electronically processing information. Although processor 12 is shown in FIG. 1 as a single entity, this is for illustrative purposes only. In some embodiments, processor 12 may comprise a plurality of processing units. These processing units may be physically located within the same device (e.g., a server), or processor 12 may represent processing functionality of a plurality of devices operating in coordination (e.g., one or more servers, computing device, devices that are part of external resources 16, electronic storage 14, or other devices.)

As shown in FIG. 1, processor 12 is configured via machine-readable instructions 24 to execute one or more computer program components. The computer program components may comprise one or more of a communications component 26, a prediction component 28, a care plan component 30, a presentation component 32, or other components. Processor 12 may be configured to execute components 26, 28, 30, or 32 by software; hardware; firmware; some combination of software, hardware, or firmware; or other mechanisms for configuring processing capabilities on processor 12.

It should be appreciated that although components 26, 28, 30, and 32 are illustrated in FIG. 1 as being co-located within a single processing unit, in embodiments in which processor 12 comprises multiple processing units, one or more of components 26, 28, 30, or 32 may be located remotely from the other components. The description of the functionality provided by the different components 26, 28, 30, or 32 described below is for illustrative purposes, and is not intended to be limiting, as any of components 26, 28, 30, or 32 may provide more or less functionality than is described. For example, one or more of components 26, 28, 30, or 32 may be eliminated, and some or all of its functionality may be provided by other components 26, 28, 30, or 32. As another example, processor 12 may be configured to execute one or more additional components that may perform some or all of the functionality attributed below to one of components 26, 28, 30, or 32.

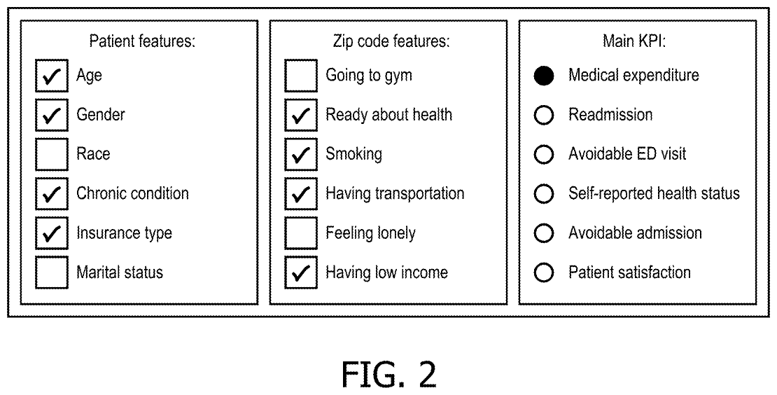

In some embodiment, the present disclosure comprises means for obtaining, (e.g., from one or more databases such as electronic storage 14, external resources 16, via information entered on user interface 20, via other resources) (i) one or more patient-related features (ii) one or more location-related features associated with an individual, (iii) one or more patient-related metrics, or (iv) other information. In some embodiments, such means for obtaining takes the form of communications component 26. In some embodiments, the patient-related features include age, gender, race, chronic condition, insurance type, marital status, or other information. In some embodiments, the location-related features include location-related features that a healthcare provider may impact via one or more care plans (described below). For example, the healthcare provider may offer a care plan (e.g., UberHEALTH) for a location-related feature corresponding to lack of transportation in an area. In some embodiments, the location-related features associated with an individual include features associated with a predetermined zip code, a plurality of neighboring zip codes, a county, a city, a state, a plurality of neighboring states, a geographic region (e.g., East Coast, West Coast), a country, a plurality of neighboring countries, or other locations associated with the individual. In some embodiments, the location-related features include one or more social determinants of health related to the individual. In some embodiments, social determinants of health include conditions in the environments in which the individual is born, living, learning, working, playing, worshiping, and age that affect a wide range of health, functioning, and quality-of-life outcomes and risks. In some embodiments, the conditions include social, economic, and physical aspects in various environments and settings (e.g., school, church, workplace, and neighborhood). In some embodiments, the patterns of social engagement and sense of security and well-being are affected by where the individual lives. In some embodiments, the social determinants of health have a significant influence on population health outcomes. Examples of these resources include safe and affordable housing, access to education, public safety, availability of healthy foods, local emergency/health services, environments free of life-threatening toxins, or other factors. Other examples of social determinants of health include going to gym, reading about health, smoking, having transportation, feeling lonely, having low income, or other location-related features. In some embodiments, the patient-related metrics include medical expenditure, readmission to a healthcare facility, avoidable emergency department visits, self-reported health status, avoidable admissions to a healthcare facility, patient satisfaction, or other patient-related metrics. By way of a non-limiting example, FIG. 2 illustrates a feature selection interface, in accordance with one or more embodiments. As shown in FIG. 2, in the left panel, a user may select the variables specific to the patient (e.g., patient-related features). In the middle panel, the user may select the location-related features (e.g., zip code-related social determinants of health factors), for which the health system has associated care plans and a potential impact on the selected patient-related metric is hypothesized by the user. In the right panel, the user may select the patient-related metrics.

Returning to FIG. 1, in some embodiment, the present disclosure comprises means for performing one or more queries on one or more databases (e.g., databases stored electronic storage 14, databases available through external resources 16, etc.) based on the patient-related features associated with the individual to obtain one or more location-related features associated with similar individuals (e.g., other individuals having patient-related features similar to the individual). In some embodiments, such means for performing takes the form of communications component 26. In some embodiments, the one or more embodiments, the one or more databases contain at least (i) one or more patient-related features and (ii) one or more location-related features associated with similar individuals. In some embodiments, the one or more databases further contain one or more patient-related features and one or more location-related features associated with other individuals. As an example, communications component 26 may obtain location-related features associated with 100 or more individuals, 1,000 or more individuals, 10,000 or more individuals, 100,000 or more individuals, 1,000,000 or more individuals, 100,000,000 or more individuals, or other number of individuals. As a use-case scenario, for example, responsive to the selection of age, gender, chronic condition, and insurance type in the left panel of FIG. 2 (for a male patient who is 70 years old with a prostate cancer and Medicare insurance), communications component 26 may obtain location-related information associated with other male patients who are 70±5 years old with a prostate cancer and Medicare insurance.

In some embodiment, the present disclosure comprises means for providing the location-related features associated with the similar individuals to a machine learning model to train the machine learning model. In some embodiments, such means for providing takes the form of prediction component 28. In some embodiments, the machine learning model is configured to make predictions related to one or more patient-related metrics. In some embodiment, the present disclosure comprises means for providing the location-related features associated with the individual to the machine learning model subsequent to the training of the machine learning model to predict (a) one or more metric values for the patient-related metrics associated with the individual and (b) at least one location-related feature associated with the individual likely to contribute to the metric values. In some embodiments, such means for providing takes the form of prediction component 28. In some embodiments, the location-related feature associated with the individual is more likely to contribute to the metric values than another location-related feature associated with the individual.

In some embodiments, prediction component 28 is configured such that training the machine learning model comprises making predictions (e.g., via k-Nearest Neighbors machine learning models or other machine learning models) related to the individual by searching through the entire training set (e.g., location-related features associated with the similar individual) for the K most similar instances (the neighbors) and summarizing the output variable for those K instances. In some embodiments, to determine which of the K instances in the training dataset are most similar to the individual, prediction component 28 is configured to utilize a distance measure (e.g., Euclidean distance). In some embodiments, predication component 30 is configured to determine the Euclidean distance as the square root of the sum of the squared differences between the individual and each of the similar individuals across all location-related features (e.g., all input attributes). In some embodiments, prediction component 28 is configured to assign weights to the contributions of the neighbors, such that the nearer neighbors contribute more to the average than the more distant ones. For example, prediction component 28 may assign each neighbor a weight of 1/d, wherein d is the distance to the neighbor.

In some embodiments, responsive to the individual identifying with a cluster (e.g., the k-nearest neighbors), prediction component 28 is configured to provide the location-related features associated with the cluster (e.g., as the training dataset) to the machine learning model or another machine learning model to further train the machine learning model. In some embodiments, prediction component 28 is configured to cause the machine learning model to make predictions related to one or more metric values for the patient-related metrics associated with each of the similar individuals. In some embodiments, prediction component 28 is configured to obtain (e.g., via communications component 26) actual values corresponding to the metric values for the patient-related metrics associated with each of the similar individuals. In some embodiments, prediction component 28 is configured to provide, subsequent to the determination of the predicted metric values for the patient-related metrics associated with each of the similar individuals, the actual values corresponding to the metric values for the patient-related metrics associated with each of the similar individuals to the machine learning model to further train the machine learning model.

In some embodiments, prediction component 28 is configured to predict, via the machine learning model, at least one location-related feature associated with the individual likely to contribute to the metric values. In some embodiments, prediction component 28 is configured to make the prediction related to the location-related feature associated with the individual likely to contribute to the metric values based on one or more outputs corresponding to the k-Nearest Neighbors machine learning model (e.g., the Euclidean distance, the weight, etc.). In some embodiments, prediction component 28 is configured to predict the metric values for the patient-related metrics associated with the individual based on the location-related feature associated with the individual likely to contribute to the metric values (e.g., such as those most likely to contribute to the metric values). In this example, prediction component 28 is configured to make the predictions related to the metric values for the patient-related metrics associated with the individual via a regression-based machine learning model (e.g., linear regression, multiple linear regression, logistic regression, etc.), a random forest-based machine learning model, or other machine learning models. In a use-case scenario, prediction component 28 is configured to select the location-related feature associated with the individual likely to contribute to the metric values (e.g., the most impactful social determinants of health for medical expenditures) for the patient-related features (e.g., age, gender, chronic condition, insurance type, etc.). In this example, prediction component 28 is configured to cause a machine learning model (e.g., Random Forest) to predict medical expenditure for all the male patients who are 70±5 years old with a prostate cancer and Medicare insurance. In this scenario, prediction component 28 may identify (i) smoking (Low/Neutral/High), (ii) having transportation (Low/Neutral/High), and (ii) having low income (Low/Neutral/High) as the most impactful (e.g., most likely to contribute) location-related features on the selected patient-related metric (e.g., medical expenditure) for all the male patients who are 70±5 years old with a prostate cancer and Medicare insurance.

In some embodiments, care plan component 30 is configured to determine, based on the predicted at least one location-related feature associated with the individual likely to contribute to the metric values, one or more care plans for the individual. In some embodiments, the care plans are configured to affect the location-related feature associated with the individual likely to contribute to the metric values by a predetermined amount. For example, care plans including (a) smoking cessation program, (b) UberHEALTH, (c) free medication program may be applicable to the above scenario. In some embodiments, responsive to the assignment of each care plan to the individual, the location-related feature associated with the individual likely to contribute to the metric values may be affected by a predetermined amount (e.g., respectively affected by the same or a different predetermined amount).

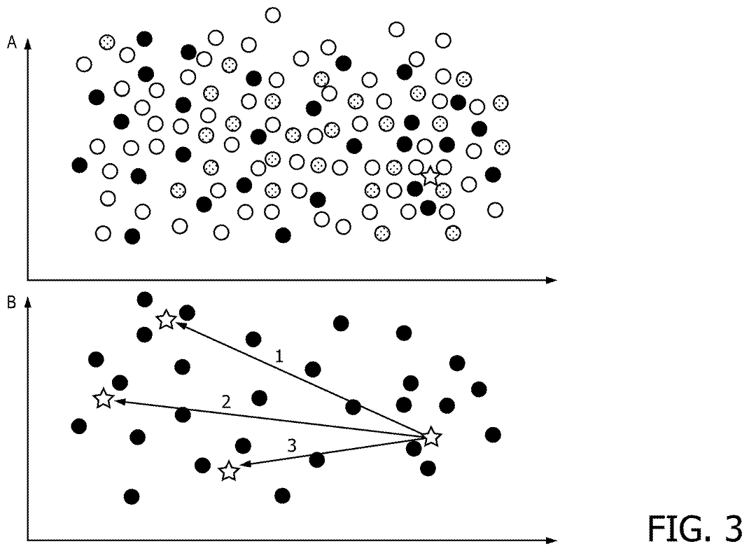

In some embodiments, care plan component 30 is configured to provide the affected at least one location-related feature associated with the individual likely to contribute to the metric values to the machine learning model to predict one or more updated metric values for the patient-related metrics associated with the individual. For example, responsive to assigning UberHEALTH to a patient living in an area with little access to public transportation, the location-related feature associated with the patient likely to contribute medical expenditures (e.g., having transportation) may be affected (e.g., the individual has access to transportation). In this example, care plan component 30 may determine (e.g., via prediction component 30) updated medical expenditures for the patient based on a new cluster of patients similar to the patient (e.g., k-nearest neighbors). By way of a non-limiting example, FIG. 3 illustrates assignment of care plans to an individual, in accordance with one or more embodiments. By way of comparison, FIG. 3A shows all patients (e.g., red, blue, and brown circles), whereas FIG. 3B shows patients (brown circles) similar to the individual (black star), in terms of patient-related features. Furthermore, as shown in FIG. 3B, the purple, cyan, and green stars are the projected situations of the individual (black star), compared to other similar patients, after assigning Care Plans 1, 2, and 3 to the individual, respectively.

Returning to FIG. 1, in some embodiments, care plan component 30 is configured to determine a change in the metric values for the patient-related metrics associated with the individual based on a difference between the previously predicted one or more metric values for the patient-related metrics associated with the individual and the updated metric values for the patient-related metrics associated with the individual. For example, responsive to the assignment of the “smoking cessation program” to the patient, the “smoking level” is subsequently changed from high to low. In this example, the change of medical expenditures (e.g., a key performance indicator) is defined as potential improvement in medical expenditures in case of assigning “smoking cessation program” to the patient. As other examples, the change of medical expenditures may be determined as potential improvement in medical expenditures in case of assigning “UberHEALTH,” “Free medication program,” or other care plans to the patient.

In some embodiments, care plan component 30 is configured to obtain (e.g., via communications component 26) costs associated with the care plans. In some embodiments, care plan component 30 is configured to determine patient-related metric improvement per unit amount (e.g., dollar amount) spent for each care plan of the care plans based on a ratio of the change in the metric values for the patient-related metrics associated with the individual and the costs associated with the corresponding care plan.

In some embodiments, care plan component 30 is configured to, responsive to the determined patient-related metric improvement per unit amount spent for each care plan of the care plans not exceeding a predetermine threshold, perform one or more iterations of the previous operations based on one or more other location-related features (e.g., a new list of social determinants of health).

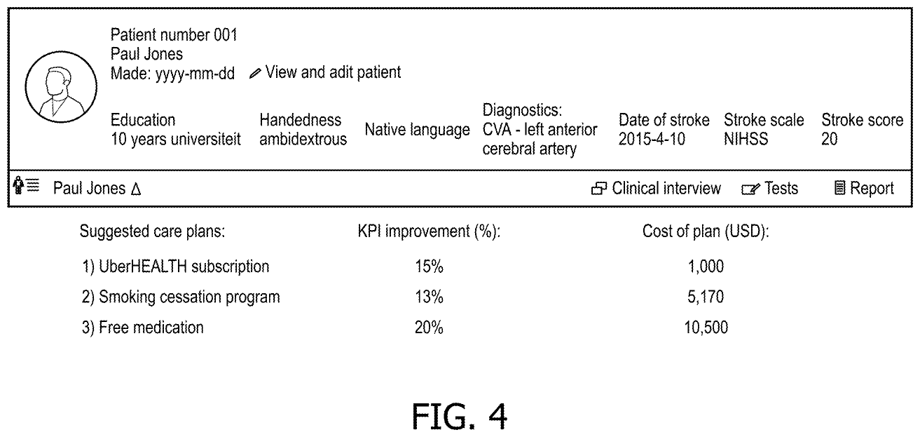

In some embodiments, presentation component 32 is configured to, responsive to the determined patient-related metric improvement per unit amount spent for each care plan of the care plans exceeding a predetermine threshold, effectuate, via user interface 20, presentation of (i) the care plan, (ii) the change in the patient-related metrics associated with the individual, (iii) the cost of the care plan, or (iv) other information. By way of a non-limiting example, FIG. 4 illustrates a care plan suggestion interface, in accordance with one or more embodiments. In FIG. 4, presentation component 32 is configured to effectuate, user interface 20, presentation of patient-specific information and suggested care plans that potentially improve the KPI along with their projected KPI improvement and corresponding cost of plan.

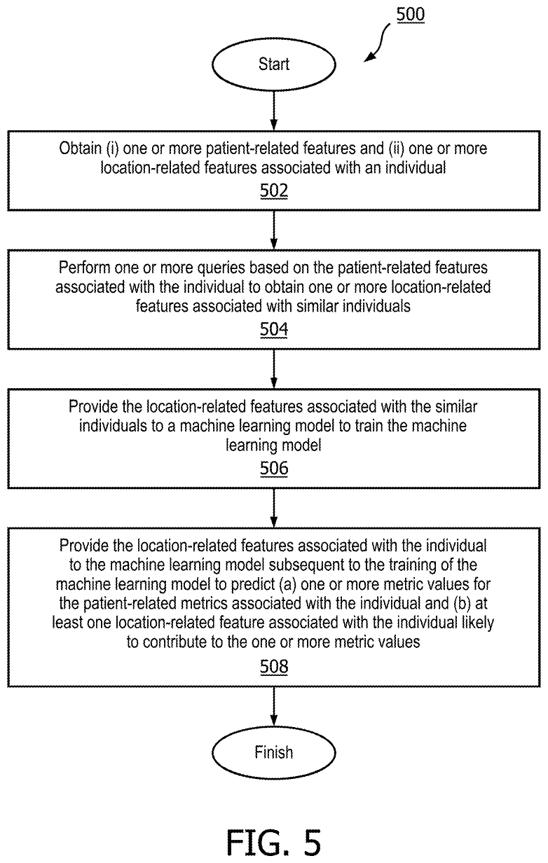

FIG. 5 illustrates a method 500 for providing model-based predictions of patient-related metrics based on location-based determinants of health, in accordance with one or more embodiments. Method 500 may be performed with a system. The system comprises one or more processors, or other components. The processors are configured by machine readable instructions to execute computer program components. The computer program components include a communications component, a prediction component, a care plan component, a presentation component, or other components. The operations of method 500 presented below are intended to be illustrative. In some embodiments, method 500 may be accomplished with one or more additional operations not described, or without one or more of the operations discussed. Additionally, the order in which the operations of method 500 are illustrated in FIG. 5 and described below is not intended to be limiting.

In some embodiments, method 500 may be implemented in one or more processing devices (e.g., a digital processor, an analog processor, a digital circuit designed to process information, an analog circuit designed to process information, a state machine, or other mechanisms for electronically processing information). The devices may include one or more devices executing some or all of the operations of method 500 in response to instructions stored electronically on an electronic storage medium. The processing devices may include one or more devices configured through hardware, firmware, or software to be specifically designed for execution of one or more of the operations of method 500.

At an operation 502, (i) one or more patient-related features and (ii) one or more location-related features associated with an individual are obtained. In some embodiments, operation 502 is performed by a processor component the same as or similar to communications component 26 (shown in FIG. 1 and described herein).

At an operation 504, one or more queries are performed on one or more databases based on the one or more patient-related features associated with the individual to obtain one or more location-related features associated with similar individuals. In some embodiments, operation 504 is performed by a processor component the same as or similar to communications component 26 (shown in FIG. 1 and described herein).

At an operation 506, the one or more location-related features associated with the similar individuals are provided to a machine learning model to train the machine learning model. In some embodiments, the machine learning model is configured to make predictions related to one or more patient-related metrics. In some embodiments, operation 506 is performed by a processor component the same as or similar to prediction component 28 (shown in FIG. 1 and described herein).

At an operation 508, the one or more location-related features associated with the individual are provided to the machine learning model subsequent to the training of the machine learning model to predict (a) one or more metric values for the patient-related metrics associated with the individual and (b) at least one location-related feature associated with the individual likely to contribute to the one or more metric values. In some embodiments, operation 508 is performed by a processor component the same as or similar to prediction component 28 (shown in FIG. 1 and described herein).

Although the description provided above provides detail for the purpose of illustration based on what is currently considered to be the most practical and preferred embodiments, it is to be understood that such detail is solely for that purpose and that the disclosure is not limited to the expressly disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment. [45] In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

What is claimed is:

1. A system for providing model-based predictions of patient-related metrics based on location-based determinants of health, the system comprising:
one or more processors configured by machine-readable instructions to:
obtain (i) one or more patient-related features and (ii) one or more location-related features associated with an individual;
perform, on one or more databases containing at least (i) one or more patient-related features and (ii) one or more location-related features associated with similar individuals, one or more queries based on the one or more patient-related features associated with the individual to obtain the one or more location-related features associated with the similar individuals;
provide the one or more location-related features associated with the similar individuals to a machine learning model to train the machine learning model, the machine learning model configured to make predictions related to one or more patient-related metrics; and
provide, subsequent to the training of the machine learning model, the one or more location-related features associated with the individual to the machine learning model to predict (a) one or more metric values for the patient-related metrics associated with the individual and (b) at least one location-related feature associated with the individual likely to contribute to the one or more metric values.

2. The system of claim 1, wherein the one or more processors are configured such that training the machine learning model comprises:
providing the one or more location-related features associated with the similar individuals to the machine learning model;
causing the machine learning model to make predictions related to the one or more patient-related metrics associated with each of the similar individuals;
obtaining actual values corresponding to the one or more patient-related metrics associated with each of the similar individuals; and
providing the actual values to the machine learning model to further train the model.

3. The system of claim 1, wherein the one or more processors are configured to:
determine, based on the at least one location-related feature associated with the individual likely to contribute to the one or more metric values, one or more care plans for the individual, the one or more care plans configured to affect the at least one location-related feature associated with the individual likely to contribute to the one or more metric values;
provide the at least one affected location-related feature associated with the individual likely to contribute to the one or more metric values to the machine learning model to predict one or more updated metric values for the patient-related metrics associated with the individual; and
determine a change in the one or more metric values for the patient-related metrics associated with the individual based on a difference between the previously predicted one or more metric values for the patient-related metrics associated with the individual and the one or more updated metric values for the patient-related metrics associated with the individual.

4. The system of claim 3, wherein the one or more processors are configured to:
obtain costs associated with the one or more care plans; and
determine patient-related metric improvement per unit amount spent for each care plan of the one or more care plans based on a ratio of the change in the one or more metric values for the patient-related metrics associated with the individual and the costs associated with the corresponding care plan.

5. The system of claim 4, wherein the one or more processors are configured to, responsive to the determined patient-related metric improvement per unit amount spent for each care plan of the one or more care plans exceeding a predetermine threshold, effectuate, via a user interface, presentation of (i) the care plan, (ii) the change in the one or more metric values for the patient-related metrics associated with the individual, and (iii) the cost of the care plan.

6. The system of claim 1, wherein the at least one location-related feature associated with the individual is more likely to contribute to the one or more metric values than another location-related feature associated with the individual.

7. A method for providing model-based predictions of patient-related metrics based on location-based determinants of health, the method comprising:
obtaining, with one or more processors, (i) one or more patient-related features and (ii) one or more location-related features associated with an individual;
performing, with the one or more processors, one or more queries on one or more databases containing at least (i) one or more patient-related features and (ii) one or more location-related features associated with similar individuals, the one or more queries being based on the one or more patient-related features associated with the individual to obtain the one or more location-related features associated with the similar individuals;
providing, with the one or more processors, the one or more location-related features associated with the similar individuals to a machine learning model to train the machine learning model, the machine learning model configured to make predictions related to one or more patient-related metrics; and
providing, with the one or more processors, the one or more location-related features associated with the individual to the machine learning model subsequent to the training of the machine learning model to predict (a) one or more metric values for the patient-related metrics associated with the individual and (b) at least one location-related feature associated with the individual likely to contribute to the one or more metric values.

8. The method of claim 7, wherein training the machine learning model comprises:
providing, with the one or more processors, the one or more location-related features associated with the similar individuals to the machine learning model;
causing, with the one or more processors, the machine learning model to make predictions related to the one or more patient-related metrics associated with each of the similar individuals;
obtaining, with the one or more processors, actual values corresponding to the one or more patient-related metrics associated with each of the similar individuals; and
providing, with the one or more processors, the actual values to the machine learning model to further train the model.

9. The method of claim 7, further comprising:
determining, with the one or more processors, one or more care plans for the individual based on the at least one location-related feature associated with the individual likely to contribute to the one or more metric values, the one or more care plans configured to affect the at least one location-related feature associated with the individual likely to contribute to the one or more metric values;
providing, with the one or more processors, the at least one affected location-related feature associated with the individual likely to contribute to the one or more metric values to the machine learning model to predict one or more updated metric values for the patient-related metrics associated with the individual; and
determining, with the one or more processors, a change in the one or more metric values for the patient-related metrics associated with the individual based on a difference between the previously predicted one or more metric values for the patient-related metrics associated with the individual and the one or more updated metric values for the patient-related metrics associated with the individual.

10. The method of claim 9, further comprising:
obtaining, with the one or more processors, costs associated with the one or more care plans; and
determining, with the one or more processors, patient-related metric improvement per unit amount spent for each care plan of the one or more care plans based on a ratio of the change in the one or more metric values for the patient-related metrics associated with the individual and the costs associated with the corresponding care plan.

11. The method of claim 10, further comprising, responsive to the determined patient-related metric improvement per unit amount spent for each care plan of the one or more care plans exceeding a predetermine threshold, effectuating, via a user interface, presentation of (i) the care plan, (ii) the change in the one or more metric values for the patient-related metrics associated with the individual, and (iii) the cost of the care plan.

12. The method of claim 7, wherein the at least one location-related feature associated with the individual is more likely to contribute to the one or more metric values than another location-related feature associated with the individual.

13. A system for providing model-based predictions of patient-related metrics based on location-based determinants of health, the method comprising:
means for obtaining (i) one or more patient-related features and (ii) one or more location-related features associated with an individual;
means for performing one or more queries on one or more databases containing at least (i) one or more patient-related features and (ii) one or more location-related features associated with similar individuals, the one or more queries being based on the one or more patient-related features associated with the individual to obtain the one or more location-related features associated with the similar individuals;
means for providing the one or more location-related features associated with the similar individuals to a machine learning model to train the machine learning model, the machine learning model configured to make predictions related to one or more patient-related metrics; and
means for providing the one or more location-related features associated with the individual to the machine learning model subsequent to the training of the machine learning model to predict (a) one or more metric values for the patient-related metrics associated with the individual and (b) at least one location-related feature associated with the individual likely to contribute to the one or more metric values.

14. The system of claim 13, wherein training the machine learning model comprises:
means for providing the one or more location-related features associated with the similar individuals to the machine learning model;
means for causing the machine learning model to make predictions related to the one or more patient-related metrics associated with each of the similar individuals;
means for obtaining actual values corresponding to the one or more patient-related metrics associated with each of the similar individuals; and
means for providing the actual values to the machine learning model to further train the model.

15. The system of claim 13, further comprising:
means for determining one or more care plans for the individual based on the at least one location-related feature associated with the individual likely to contribute to the one or more metric values, the one or more care plans configured to affect the at least one location-related feature associated with the individual likely to contribute to the one or more metric values;
means for providing the at least one affected location-related feature associated with the individual likely to contribute to the one or more metric values to the machine learning model to predict one or more updated metric values for the patient-related metrics associated with the individual; and
means for determining a change in the one or more metric values for the patient-related metrics associated with the individual based on a difference between the previously predicted one or more metric values for the patient-related metrics associated with the individual and the one or more updated metric values for the patient-related metrics associated with the individual.

16. The system of claim 15, further comprising:
means for obtaining costs associated with the one or more care plans; and
means for determining patient-related metric improvement per unit amount spent for each care plan of the one or more care plans based on a ratio of the change in the one or more metric values for the patient-related metrics associated with the individual and the costs associated with the corresponding care plan.

17. The system of claim 16, further comprising, responsive to the determined patient-related metric improvement per unit amount spent for each care plan of the one or more care plans exceeding a predetermine threshold, means for effectuating presentation of (i) the care plan, (ii) the change in the one or more metric values for the patient-related metrics associated with the individual, and (iii) the cost of the care plan.
